(19) United States
(12) Patent Application Publication
(10) Pub. No.: US 2024/0216189 A1
(43) Pub. Date: Jul. 4, 2024

(54) ABSORBENT ARTICLE BARRIER LAYER

(71) Applicant: JOHNSON & JOHNSON CONSUMER INC., Skillman, NJ (US)

(72) Inventors: Aline Pereira, São Jose dos Campos/SP (BR); Gilson Sardinha, São Paulo (BR)

(21) Appl. No.: 18/541,903

(22) Filed: Dec. 15, 2023

Related U.S. Application Data
(60) Provisional application No. 63/477,599, filed on Dec. 29, 2022.

(51) Int. Cl. A61F 13/514; A61F 13/15; A61F 13/47

(52) U.S. Cl. CPC A61F 13/51456; A61F 13/15707; A61F 13/47

(57) ABSTRACT

An absorbent article comprising a liquid permeable cover layer, an impervious barrier layer and an absorbent core between the cover layer and the barrier layer, wherein the barrier layer is impervious and biodegradable and comprises a kraft paper layer and a continuous resin coating; wherein the continuous resin coating is on the surface of the barrier layer disposed toward the absorbent core, and the continuous resin coating weight is from 10 to 18 g/m², and the continuous resin coating is an acrylic copolymer.

### CROSS-REFERENCE TO RELATED APPLICATIONS

This application claims priority to U.S. Provisional Application Ser. No. 63/477,599 filed Dec. 29, 2022, the entire contents of which is hereby incorporated by reference herein.

### TECHNICAL FIELD

The invention generally relates to barrier layer and more particularly to absorbent article with a barrier layer. In particular, the invention relates to an absorbent article comprising a liquid permeable cover layer, an impervious barrier layer and an absorbent core between the cover layer and the barrier layer, wherein the barrier layer is impervious and biodegradable and comprises a kraft paper layer and a continuous resin coating. The invention also relates to a liquid impervious barrier layer comprising a kraft paper layer and a continuous resin coating. The invention also relates to a method of manufacturing said absorbent article with a barrier layer comprising a kraft paper layer and a continuous resin coating. The invention also relates to the use of a barrier layer comprising a kraft paper layer and a continuous resin coating, for the construction of an absorbent article.

### BACKGROUND

Disposable absorbent articles such as pantiliners, sanitary napkins, interlabial devices, adult incontinence devices, diapers, and nursing pads are well known in the art. These articles typically have a fluid permeable body-facing side (or cover layer) and fluid impermeable garment facing side (or barrier layer). Additionally, such articles may include an absorbent layer (or absorbent core) for retaining fluids therebetween.

Environmental concerns are driving consumers to prefer more sustainable absorbent article with a higher proportion of renewable materials, or less materials petrochemically derived, in their composition. Often the barrier layer of absorbent articles is made of plastic materials such as polyethylene. Petrochemically sourced adhesives may also be used in traditional absorbent articles, either for holding elements in place while the absorbent article is manufactured (construction adhesives), or to hold the absorbent article in place over the undergarment while in use by the consumer (positioning adhesives).

Patent application US2006/0154054 by John Banks purports to disclose a flushable bodily liquid absorbent composite product, having a bodily liquid absorbent core and a backing layer applied to a garment side of the core. The backing layer is readily soluble in cold water and has a water impervious layer on its core side and a water-resistant layer on its garment facing side. Backing layer is made of polyvinyl acetate.

U.S. Pat. No. 3,561,447 by Kendall & Co purports to disclose a flushable sanitary napkin and a non-woven fabric to cover said napkin. The web of textile length fibers being unified by a mixture of acrylic polymeric binder and polyvinyl alcohol.

Patent application EP0059503 by Procter & Gamble purports to disclose a disposable diaper provided with a two-element breathable back sheet. The two elements are a vapor pervious, relatively liquid impervious outer sheet and a liquid impervious inner panel. An absorbent core is the third necessary element of the disposable diaper. The inner panel is placed between the outer sheet and the absorbent core in the crotch region of the disposable diaper.

These prior arts propose flushable alternatives to plastic sourced barrier layers. However, they are not entirely satisfactory since the used material may not be completely renewable. Reduction or absence of adhesives is also not addressed.

Therefore, there is a need to propose a more sustainable alternative to conventional absorbent article by providing a new type of barrier layer. There is also a need to find a solution to reduce and avoid, if possible, the use of adhesive in the absorbent article.

It has surprisingly been found that both those needs may be addressed by implementing a barrier layer according to the present invention in an absorbent article.

### SUMMARY

One aspect of the invention pertains to an absorbent article comprising a liquid permeable cover layer, an impervious barrier layer and an absorbent core between the cover layer and the barrier layer, wherein the barrier layer is impervious and biodegradable and comprises a kraft paper layer and a continuous resin coating; wherein the continuous resin coating is on the surface of the barrier layer disposed toward the absorbent core, and the continuous resin coating weight is from 10 to 18 g/m², preferably 12 to 15 g/m², and the continuous resin coating is an acrylic copolymer.

In one embodiment, the kraft paper layer comprises 80% to 50% of cellulosic fibers having an average length greater than about 2 mm, preferably between about 2 mm and about 5 mm. In some embodiments the kraft paper layer has a basis weight from 28 to 32 g/m². In one or more embodiment the kraft paper layer has a thickness from 45 to 60 μm. In one or more embodiment the kraft paper layer has a transversal tear resistance superior to 25 gf. In some embodiment the kraft paper layer does not comprises a Polyethylene coating layer or a plant-based protein coating layer. In one or more embodiment the kraft paper layer is unbleached.

In some embodiments the absorbent article does not comprise a construction adhesive between the absorbent core and the continuous resin coating of the barrier layer. In one or more embodiment a transfer layer is present between the cover layer and the absorbent core. In one or more embodiment the liquid permeable cover layer is manufactured from at least 70% of biodegradable fibers or compostable fibers, such as natural fibers.

In some embodiments the absorbent article does not comprise a positioning adhesive on the face of the kraft paper layer opposite to the continuous resin coating.

Another aspect of the invention pertains to a liquid impervious barrier layer comprising a kraft paper layer and a continuous resin coating; wherein the continuous resin coating weight is from 10 to 18 g/m², preferably 12 to 15 g/m², and the continuous resin coating is an acrylic copolymer, wherein the thickness of the barrier layer ranges from about 40 μm to about 80 μm.

Another aspect of the invention pertains to a process for the construction of an absorbent article comprising the following steps: applying a continuous resin coating on a kraft paper layer, to obtain a resin coated kraft paper; drying the resin coated kraft paper in an oven to obtain a barrier layer; assembling the barrier layer with at least a liquid permeable cover layer and optionally an absorbent core between the barrier layer and the cover layer to obtain an absorbent article, wherein the continuous resin coating weight is from 10 to 18 g/m², preferably 12 to 15 g/m², and the continuous resin coating is an acrylic copolymer.

The application step of the continuous resin coating on the kraft paper layer may be done through processes known to those of ordinary skill in the art. Often, one of skill in the art will select either gravure or flexography processes. For example, gravure processes such as rotogravure processes can produce high-quality continuous coatings, desirable to provide leak-proof barrier materials.

In some embodiments the application step of the continuous resin coating on the kraft paper layer is done in at least two distinct steps; preferably in 2 distinct steps, before the resin coated kraft paper is dried, and wherein each coating layer has a weight of 06 to 09 g/m².

Another aspect of the invention pertains to the use of a liquid impervious barrier layer comprising a kraft paper layer and a continuous resin coating; wherein the continuous resin coating weight is from 10 to 18 g/m², preferably 12 to 15 g/m², and the continuous resin coating is an acrylic copolymer, wherein the thickness of the barrier layer ranges from about 40 μm to about 80 μm, for the construction of an absorbent article such as a feminine hygiene product, for example a napkin, a panty liner.

### BRIEF DESCRIPTION OF THE DRAWING

FIG. 1 is top plan view of an exemplary absorbent article according to the present invention.

FIG. 2 is a cross-section along line X-X of a first embodiment of the present invention.

FIG. 3 is a cross-section along line X-X of a second embodiment of the present invention.

FIG. 4 is a cross-section along line X-X of a third embodiment of the present invention.

FIG. 5 is a cross-section along line X-X of a fourth embodiment of the present invention.

FIG. 6 is a cross-section along line X-X of a fifth embodiment of the present invention.

FIG. 7 is a cross-section along line X-X of a sixth embodiment of the present invention.

FIG. 8 is a schematic illustration of a process according to the present invention.

FIG. 9 is a cross-section along line X-X of a seventh embodiment of the present invention.

### DETAILED DESCRIPTION OF THE INVENTION

The terms used in this specification generally have their ordinary meanings in the art within the context of this disclosure and in specific context where each term is used. Certain terms are discussed below, or elsewhere in the specification, to provide additional guidance in describing the compositions and methods of the disclosure and how to make and use them.

As used in the specification and the appended claims, the singular forms “a,” “an,” and “the” include the plural referents unless the context clearly dictates otherwise. Thus, for example, reference to “an ingredient” includes mixtures of ingredients.

As used herein, the terms “about” or “approximately” mean within an acceptable error range for the particular value as determined by one of ordinary skill in the art, which will depend in part on how the value is measured or determined (i.e., the limitations of the measurement system). For example, “about” can mean within three or more standard deviations per practice in the art. Alternatively, “about” can mean a range of up to 10%, up to 5%, up to 3%, up to 1%, or up to 0.5% of a given value. Particularly with respect to systems or processes, the term can mean within an order of magnitude, for example, within five-fold or within two-fold of a value.

As used herein, the terms “% w/w” or “weight percent” refers to the percentage of an ingredient(s)/the total percentage by weight of the composition (100%). The terms “% w/w” or “weight percent” refer to the quantity by weight of a constituent or component, for example, in a continuous resin coating as a percentage of the overall weight of the continuous resin coating. The terms “weight percent”, “wt-%”, “wt. %”, and “wt %” are used interchangeably.

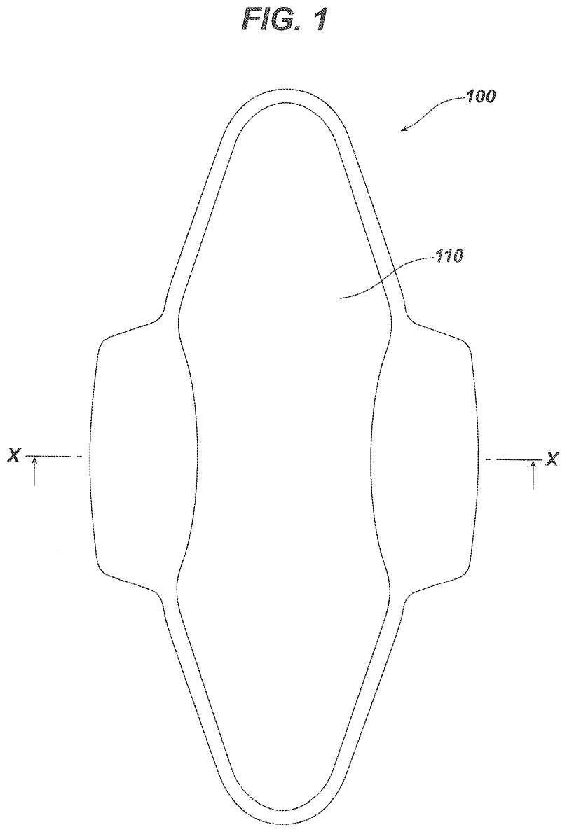

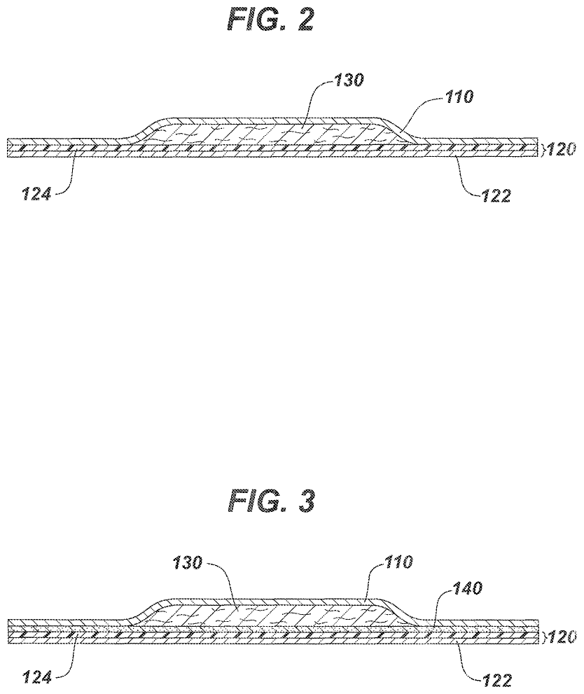

One embodiment of the invention relates to an absorbent article, such as a sanitary napkin 100 shown in plan view in FIGS. 1 and 2, comprising a liquid permeable cover layer 110, an impervious barrier 120 and an absorbent core 130 between the cover layer 110 and the barrier 120, wherein the barrier 120 is impervious and biodegradable and comprises a kraft paper layer 122 and a continuous resin coating 124, wherein the continuous resin coating 124 defines the surface 126 of the kraft paper layer 122 disposed toward the absorbent core 130, and the continuous resin coating weight is present from about 10 to about 18 g/m², preferably about 12 to about 15 g/m² on the kraft paper layer 122, and the continuous resin coating 124 is an acrylic copolymer.

If the amount of continuous resin coating 124 on the kraft paper layer 122 is too low, for example below 10 g/m², the resulting barrier 120 may not be properly impervious to fluids collected by the absorbent article 100 and some of the fluids may leak through the barrier. On the contrary, if the coating layer 124 is too thick, for example above 18 g/m², the barrier 120 may stick to itself when rolled on a coil for storage and transportation, thus causing tearing or delamination of the barrier 120 when unrolling during manufacture of the absorbent article 100.

By “kraft paper” it is meant a paper produced in the kraft process. Kraft process is a process for conversion of wood into wood pulp, which consists of almost pure cellulose fibers, the main component of paper. The kraft process involves treatment of wood chips with a hot mixture of water, sodium hydroxide (NaOH), and sodium sulfide (Na₂S).

The continuous resin coating may be applied to the kraft paper as an aqueous solution comprising from about 45% to about 70% of water, preferably about 55% to about 60% of water, in weight %.

In one embodiment of the absorbent article, the kraft paper layer 122 may comprise a blend of cellulosic fibers, from 80% to 50% of cellulosic fibers having an average length greater than about 2 mm.

Preferably, the cellulosic fibers may have an average length from about 2 mm to about 5 mm.

The fiber length described above provides more tensile strength to the kraft paper layer 122 and therefore enables more robust production processes for the barrier 120 and for the absorbent article 100.

The above fiber blend may be provided by blending relatively short fibers derived from trees of the Eucalyptus family and relatively long fibers derived from trees of the Pine family.

In some embodiments of the absorbent article 100, the kraft paper layer 122 may have a basis weight from 28 to 32 g/m².

In one or more embodiment of the absorbent article 100, the kraft paper layer 122 may have a thickness from 45 to 60 μm.

In one or more embodiment of the absorbent article 100, the kraft paper layer 122 may have a transversal tear resistance superior to 25 gf, preferably superior to 28 gf, or preferably superior or equal to 30 gf.

Transversal tear resistance is a desirable property for kraft paper layer 122 in the context of the present invention since it allows for a more robust production process of the barrier 120 and for the absorbent article 100.

In one or more embodiment of the absorbent article 100, the kraft paper layer 122 may not comprise a polyethylene coating layer, a polylactic acid coating layer or a plant-based protein coating layer.

Plant based protein coating such as zein coating used in food industry may not be part of the scope of the present invention. Polyethylene coating layer may not be part of the present invention as they may be considered not biodegradable.

In one or more embodiment of the absorbent article the barrier 120 may consist essentially of a kraft paper layer 122 and a continuous resin coating 124.

In one or more embodiment of the absorbent article, the kraft paper layer 122 may be unbleached.

In a particular embodiment, the kraft paper layer 122 may be white.

In one or more embodiment of the absorbent article, there may be no construction adhesive between the absorbent core 130 and the continuous resin coating 124 of the barrier 120.

Reduction of the construction adhesive is a desirable feature to reduce costs of production and to propose more sustainable products to consumers. In the present invention the continuous resin coating 124 may assist in attaching elements of the absorbent article 100 together in addition to its function to render the kraft paper impervious to liquids.

In a particular embodiment, the absorbent article may consist essentially of a liquid permeable cover layer 110, an impervious barrier 120 and an absorbent core 160 between the cover layer 110 and the barrier 120, wherein the barrier 120 is impervious and biodegradable and comprises a kraft paper layer 122 and a continuous resin coating 124; wherein the continuous resin coating 124 is on the surface 126 of the kraft paper layer 122 disposed toward the absorbent core 130, and the continuous resin coating weight is from 10 to 18 g/m², preferably 12 to 15 g/m², and the continuous resin coating is an acrylic copolymer.

In a particular embodiment, the absorbent article 100 may consist essentially of a liquid permeable cover layer 110, an impervious barrier 120 and an absorbent core 130 between the cover layer 110 and the barrier 120, wherein the barrier 120 is impervious and biodegradable and consists essentially of a kraft paper layer 122 and a continuous resin coating 124; wherein the continuous resin coating 124 is on the surface 126 of the kraft paper layer 122 disposed toward the absorbent core 130, and the continuous resin coating weight is from 10 to 18 g/m², preferably 12 to 15 g/m², and the continuous resin coating is an acrylic copolymer.

In another particular embodiment shown in FIG. 3-5, the absorbent article may consist essentially of a liquid permeable cover layer 110, an impervious barrier 120, an absorbent core 130 between the cover layer 110 and the barrier 120, and a construction adhesive 140, wherein the barrier 120 is impervious and biodegradable and comprises a kraft paper layer 122 and a continuous resin coating 124; wherein the continuous resin coating 124 is on the surface 126 of the kraft paper layer 122 disposed toward the absorbent core 130, and the construction adhesive 140 is present between the absorbent core 130 and the continuous resin coating 122 of the barrier 120 (FIG. 3), and the continuous resin coating weight is from 10 to 18 g/m², preferably 12 to 15 g/m², and the continuous resin coating is an acrylic copolymer.

In one or more embodiment the absorbent article 100 may comprise a reduced amount of construction adhesive 180 compared to current product. A typical absorbent article such as a napkin may comprise from 5 to 5.5 g/m² of construction adhesive.

By “construction adhesive” it is meant an adhesive component functioning to attach elements of the absorbent article together.

The absorbent article 100 may comprise less than 4 g/m², preferably, less than 3 g/m², preferably less than 2.5 g/m², preferably less than 2 g/m², preferably less than 1.5 g/m², of construction adhesive 140 in the absorbent article.

The absorbent article may comprise from about 1 g/m² to about 4 g/m², preferably from about 1.25 g/m² to about 3 g/m², from about 1.25 g/m² to about 2 g/m², from about 1.25 g/m² to about 1.5 g/m² of construction adhesive.

Therefore, the overall reduction of construction adhesive in the absorbent article may be about 50%, about 60%, about 70%, about 75%, about 80%, about 90%.

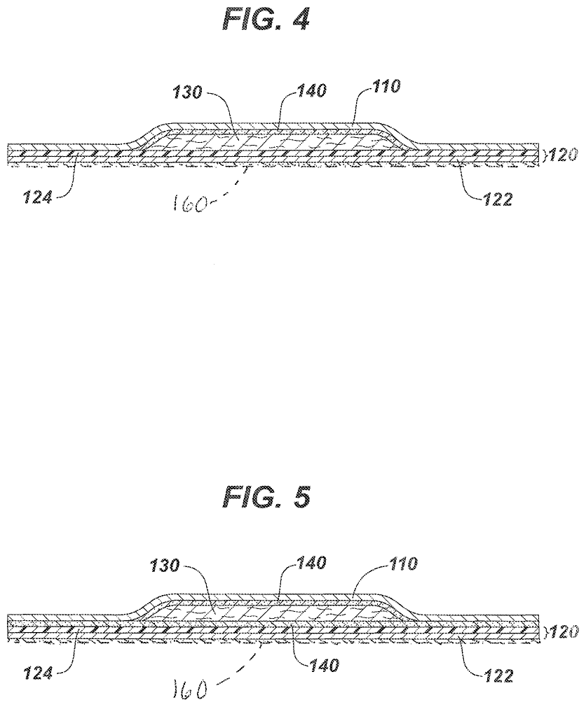

The construction adhesive 140 may be present between the cover layer 110 and the absorbent core 130 (FIG. 4), or between the barrier 120 and the absorbent core 130 (FIG. 3); or both between the cover layer 110 and the absorbent core 130 and between the barrier 120 and the absorbent core 130 (FIG. 5).

The construction adhesive is applied to the elements of the absorbent article in a discontinuous pattern to permit bodily fluids to pass therethrough, e.g. between the cover 110 to the absorbent layer 130. This may be achieved by applying a fine spray pattern, a spiral spray or jet pattern or other methods known to those of ordinary skill in the art.

In one or more embodiment of the absorbent article, a transfer layer may be present between the cover layer and the absorbent core.

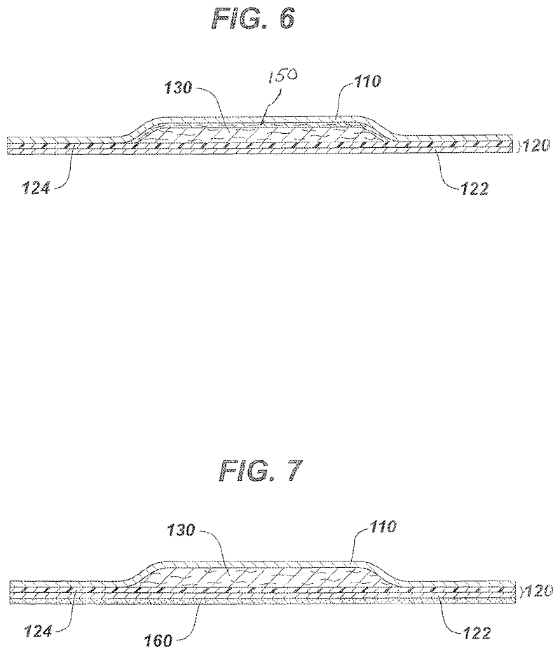

In another particular embodiment shown in FIG. 6, the absorbent article 100 may consist essentially of a liquid permeable cover layer 110, an impervious barrier 120, a transfer layer 150 and an absorbent core 130 between the transfer layer 150 and the barrier 120, wherein the barrier 120 is impervious and biodegradable and comprises a kraft paper layer 122 and a continuous resin coating 124, wherein the continuous resin coating 122 is on the surface 126 of the kraft paper layer 124 disposed toward the absorbent core 130, and the transfer layer 150 is present between the cover layer 110 and the absorbent core 130, and the continuous resin coating 122 weight is from 10 to 18 g/m², preferably 12 to 15 g/m², and the continuous resin coating is an acrylic copolymer.

In one or more embodiment of the absorbent article, the liquid permeable cover layer 110 may be manufactured from at least 70% of biodegradable fibers or compostable fibers, such as natural fibers.

By “biodegradable” it is meant the fibers may be broken down by microorganisms, such as bacteria and fungi, to generate organic matter. Composting is a human-driven process in which biodegradation occurs under a specific set of circumstances.

The liquid permeable cover layer 110 may be manufactured from at least 80%, preferably at least 90%, preferably at least 95%, preferably 100%, of biodegradable fibers or compostable fibers.

In one or more embodiment of the absorbent article 100, there is no positioning adhesive on the face of the kraft paper layer 122 opposite to the continuous resin coating 124.

In one or more embodiment the absorbent article 100 may comprise a reduced amount of positioning adhesive 160 compared to current product (See e.g., FIG. 7. A typical absorbent article 100 such as a napkin may comprise from 18.1 to 18.5 g/m² of positioning adhesive 160.

By “positioning adhesive” it is meant an adhesive placed on the garment facing side of the barrier for holding the absorbent product in place while in use.

The absorbent article may comprise less than 15 g/m², preferably, less than 12 g/m², preferably less than 8 g/m², preferably less than 6 g/m², preferably less than 5 g/m², of positioning adhesive on the absorbent article kraft paper layer opposite to the continuous resin coating.

The absorbent article may comprise from about 1 g/m² to about 15 g/m², preferably from about 2 g/m² to about 12 g/m², from about 2 g/m² to about 5 g/m², from about 4 g/m² to about 12 g/m² of positioning adhesive on the absorbent article kraft paper layer opposite to the continuous resin coating.

Overall reduction of positioning adhesive in the absorbent article may be about 40%, about 50%, about 60%, about 70%, about 75%, about 80%, about 90%.

In another particular embodiment the absorbent article 100 may consist essentially of a liquid permeable cover layer 110, an impervious barrier 120, an absorbent core 130 between the cover layer 110 and the barrier 120, and a positioning adhesive 160, wherein the barrier 120 is impervious and biodegradable and comprises a kraft paper layer 122 and a continuous resin coating 124, wherein the continuous resin coating 124 is on the surface 126 of the kraft paper layer 122 disposed toward the absorbent core 130, and the continuous resin coating weight is from 10 to 18 g/m², preferably 12 to 15 g/m², and the continuous resin coating is an acrylic copolymer, and the positioning adhesive 160 is present on the barrier 120 side opposite to the absorbent core 130.

Another embodiment of the invention relates to a liquid impervious barrier 120 comprising a kraft paper layer 122 and a continuous resin coating 124; wherein the continuous resin coating weight is from 10 to 18 g/m², preferably 12 to 15 g/m², and the continuous resin coating is an acrylic copolymer, wherein the thickness of the barrier layer ranges from about 40 μm to about 80 μm, preferably from about 45 μm to about 70 μm, preferably from about 45 μm to about 60 μm.

The continuous resin coating of the barrier layer may be lipid resistant.
By “lipid resistant” it is meant that the continuous resin coating is not soluble in lipids.
The continuous resin coating may be an aqueous solution comprising from about 45% to about 70% of water, preferably about 55% to about 60% of water, in weight %.

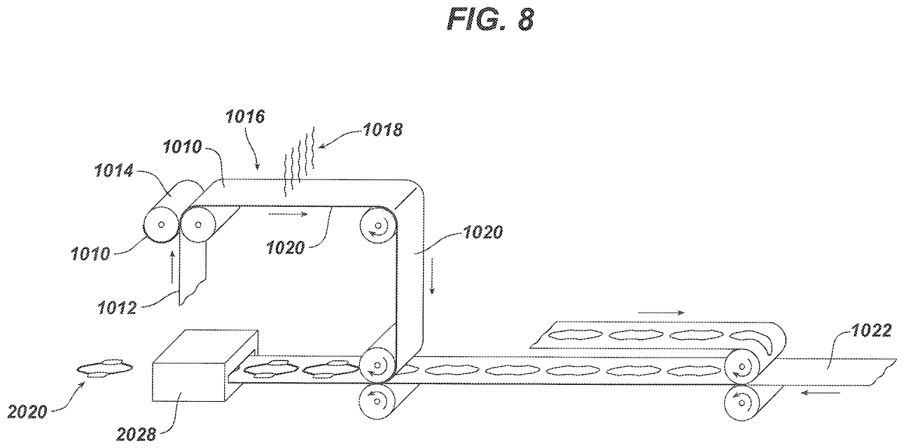

Another embodiment of the invention, shown in FIG. 8, relates to a process for the construction of absorbent articles as disclosed above, comprising the following steps:
Applying a continuous resin coating 1010 on a kraft paper layer 1012 (e.g., by rotogravure or flexographic printing roller 1014), to obtain a continuous resin coated kraft paper material 1016,
Drying the resin coated kraft paper material 1016 in an oven 1018 to obtain a continuous barrier material 1020,
Assembling the continuous barrier material 1020 with at least a continuous liquid permeable cover material 1022 and optionally discrete absorbent core segments 1024 between the barrier material 1020 and the cover material 1022, and
Separating individual absorbent articles 1026 from the assembled continuous barrier material, optional absorbent core segments and continuous cover material (e.g., in a finishing station 2028, wherein the continuous resin coating weight is from 10 to 18 g/m², preferably 12 to 15 g/m², and the continuous resin coating is an acrylic copolymer.

In a particular embodiment the drying step of the resin coated kraft paper may comprise an oven temperature ranging from 120° C. to 150° C., preferably 130° C. to 140° C.

In a particular embodiment the process for the construction of an absorbent article may comprise an additional crimping step to close off the edges of the absorbent article. The crimping step occurs after the assembling step, e.g., in the finishing station.

In one or more embodiment of the process for the construction the absorbent article, the application step of the continuous resin coating on the kraft paper layer may be done in a single coating pass before the resin coated kraft paper is dried.

In one or more embodiment of the process for the construction the absorbent article, the application step of the continuous resin coating on the kraft paper layer may be done in at least two distinct coating passes; preferably in 2 distinct coating passes, before the resin coated kraft paper is dried.

In a particular embodiment the process for the construction of an absorbent article may comprise an additional step of applying a construction adhesive to the resin coated side of the barrier layer, before assembling the barrier layer with at least a liquid permeable cover layer.

In a particular embodiment the application step of the continuous resin coating on the kraft paper layer may be done in two distinct steps, wherein each steps provides a coating layer having a weight of 06 to 09 g/m².

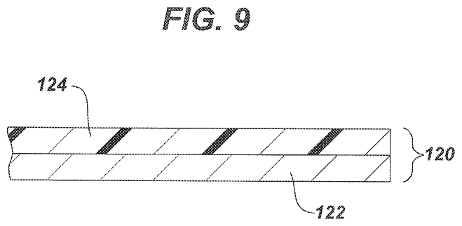

Another embodiment of the invention, shown in FIG. 9, relates to the use of a liquid impervious barrier layer comprising a kraft paper layer and a continuous resin coating, for the construction of an absorbent article such as a feminine hygiene product, for example a napkin, a panty liner, wherein the continuous resin coating weight is from 10 to 18 g/m², preferably 12 to 15 g/m², and the continuous resin coating is an acrylic copolymer, wherein the thickness of the barrier layer ranges from about 40 μm to about 80 μm.

### Examples

### Example 1: Barrier Layer

### Material:

Continuous resin coating:
Cartaseal SCK resin from Archroma. Aqueous acrylic copolymer resin, about 42% solid content.

### Paper:

850 mm wide paper coil
Monolucent white paper 30 g/m² from INAPEL, short fibers (from about 0.2 mm to about 2 mm).
Brown Kraft paper 30 g/m² from INAPEL, long fibers (from about 2 mm to about 5 mm).

### Process:

Resin is applied on paper coil by flexographic printing, in one or two layers.
Paper is dried in an oven at temperature: 130 to 140° C.

Applications of 3 different weights of resin 12.5 g/m², 14 g/m² or 18 g/m², were performed on 2 paper samples: Monolucent white paper 30 g/m² and Brown Kraft paper 30 g/m².

For the brown Kraft paper application of resin was made on the rough face of the paper.

Application in 2 layers allows for a better control of the coating thickness, surface smoothness and material tensile strength; for example, about 7 g/m² and about 8 g/m² in order to obtain a final coating layer of about 14 g/m². It is preferred to have a first layer not dry before applying the second layer.

Preferred barrier layer samples were obtained with a 14 g/m² continuous resin coating.

Trials of absorbent article construction were preformed using both paper with a 14 g/m² continuous resin coating.

### Example 2: Absorbent Article

### Material

Absorbent core: Disintegrated cellulose pulp and superabsorbent polymer (Sodium Polyacrylate)
Cover layer: non-woven polyethylene

Absorbent article length: about 230 mm width: about 140 mm; weight: about 5.5 g (wherein the absorbent core amounts for approximately 4 g); thickness: from 3.5 to 5 mm

### Standard Amount of Construction Adhesive:

Construction adhesive is located:
   Between cover layer and absorbent core, standard average construction adhesive grammage is from about 2 to 2.3 g/m².
   Between barrier layer and absorbent core, standard average construction adhesive grammage is from 3 to 3.2 g/m².

### Standard Amount of Positioning Adhesive:

Positioning adhesive is located on the side of barrier layer facing the undergarment, standard average positioning adhesive grammage is from 18.1 to 18.5 g/m².

### Trial 1

### Barrier Layer Sample:

Monolucent white paper 30 g/m² from INAPEL, short fibers, coated with Cartaseal Resin SCK from Archroma 14 g/m².
Barrier layer sample width of 160 mm.
   Two Prototypes with Paper Barrier Layer were Obtained:
1) 75% reduction of construction adhesive (from about 1.25 to 1.37 g/m²) and 75% reduction of positioning adhesive (from about 4.55 to 4.65 g/m²).
2) 75% reduction of construction adhesive (from about 1.25 to 1.37 g/m²) and 40% reduction of positioning adhesive (from about 10.8 to 11.1 g/m²).

Production was possible but issues occurred. The paper barrier layer was too fragile to resist the tension applied during manufacturing and tearing of the paper barrier layer occurred.

### Trial 2

### Barrier Layer Sample:

Brown Kraft paper 30 g/m² from INAPEL, long fibers, coated with Cartaseal SCK from Archroma 14 g/m² resin. Resin applied in two layers.
Barrier layer sample width of 160 mm.
   Three Prototypes with Brown Kraft Paper Barrier were Obtained:
1) 75% reduction of construction adhesive (from about 1.25 to 1.37 g/m²) and 50% reduction of positioning adhesive (from about 9 to 9.25 g/m²).
2) 75% reduction of construction adhesive (from about 1.25 to 1.37 g/m²) and 60% reduction of positioning adhesive (from about 7.2 to 7.4 g/m²).
3) 70% reduction of construction adhesive (from about 1.5 to 1.65 g/m²) and 60% reduction of positioning adhesive (from about 7.2 to 7.4 g/m²).
No failure of the production line was observed, no tearing, no alignment/phasing problem.

1. Absorbent article comprising a liquid permeable cover layer, an impervious barrier layer and an absorbent core between the cover layer and the barrier layer, wherein the barrier layer is impervious and biodegradable and comprises a kraft paper layer and a continuous resin coating,
wherein the continuous resin coating is on the surface of the barrier layer disposed toward the absorbent core, and
the continuous resin coating weight is from 10 to 18 g/m², preferably 12 to 15 g/m², and the continuous resin coating is an acrylic copolymer.

2. Absorbent article according to claim 1, wherein the kraft paper layer comprises 80% to 50% of cellulosic fibers having an average length greater than about 2 mm.

3. Absorbent article according to claim 1, wherein the kraft paper layer has a basis weight from 28 to 32 g/m².

4. Absorbent article according to claim 1, wherein the kraft paper layer has a thickness from 45 to 60 μm.

5. Absorbent article according to claim 1, wherein the kraft paper layer has a transversal tear resistance greater than 25 gf.

6. Absorbent article according to claim 1, wherein the kraft paper layer does not comprises a polyethylene coating layer, a polylactic acid coating layer or a plant-based protein coating layer.

7. Absorbent article according to claim 1, wherein the kraft paper layer is unbleached.

8. Absorbent article according to claim 1, wherein there is no construction adhesive between the absorbent core and the continuous resin coating of the barrier layer.

9. Absorbent article according to claim 1, wherein a transfer layer is present between the cover layer and the absorbent core.

10. Absorbent article according to claim 1, wherein the liquid permeable cover layer is manufactured from at least 70% of biodegradable fibers or compostable fibers, such as natural fibers.

11. Absorbent article according to claim 1, wherein there is no positioning adhesive on the face of the kraft paper layer opposite to the continuous resin coating.

12. Liquid impervious barrier layer comprising a kraft paper layer and a continuous resin coating; wherein the continuous resin coating weight is from 10 to 18 g/m², preferably 12 to 15 g/m², and the continuous resin coating is an acrylic copolymer, wherein the thickness of the barrier layer ranges from 40 μm to 80 μm.

13. Process for the construction of an absorbent article comprising the following steps:
Applying a continuous resin coating on a kraft paper layer, to obtain a resin coated kraft paper,
Drying the resin coated kraft paper in an oven to obtain a barrier layer,
Assembling the barrier layer with at least a liquid permeable cover layer and optionally an absorbent core between the barrier layer and the cover layer to obtain an absorbent article,
wherein the continuous resin coating weight is from 10 to 18 g/m², preferably 12 to 15 g/m², and the continuous resin coating is an acrylic copolymer.

14. Process for the construction of an absorbent article according to claim 13, wherein the application step of the continuous resin coating on the kraft paper layer is done in at least two distinct steps.

15. Use of a liquid impervious barrier layer comprising a kraft paper layer and a continuous resin coating, for the construction of an absorbent article such as a feminine hygiene product, for example a napkin, a panty liner;
wherein the continuous resin coating weight is from 10 to 18 g/m², preferably 12 to 15 g/m², and the continuous resin coating is an acrylic copolymer, wherein the thickness of the barrier layer ranges from about 40 μm to about 80 μm.
